(19) United States
(12) Patent Application Publication
(10) Pub. No.: US 2022/0072564 A1
(43) Pub. Date: Mar. 10, 2022

(54) ROTOR FOR CENTRIFUGE AND CENTRIFUGE COMPRISING THE SAME

(71) Applicant: CYTODX INC., Daejeon (KR)

(72) Inventors: Joseph SUNOO, Seongnam-si (KR); Jaephil DO, Seoul (KR); Min Pyo HONG, Suwon-si (KR)

(73) Assignee: CYTODX INC., Daejeon (KR)

(21) Appl. No.: 17/469,268

(22) Filed: Sep. 8, 2021

(30) Foreign Application Priority Data
Sep. 9, 2020 (KR) 10-2020-0115302

(51) Int. Cl. B04B 5/04; B04B 9/02; G01N 1/40

(52) U.S. Cl. CPC B04B 5/0442; G01N 1/4077; B04B 9/02

(57) ABSTRACT

A rotor for a centrifuge includes a main body to rotate about a rotating axis extending in a vertical direction, a first chamber coupled to the main body to be rotatable about a first axis perpendicular to a direction parallel to the vertical direction, and having a first space to receive a material, and a second chamber coupled to the main body to be rotatable about a second axis parallel to the first axis and having a second space to receive a material. The first space and the second space have mutually different sizes, and the center of mass of the rotor is positioned on the rotating axis.

CROSS-REFERENCE TO RELATED APPLICATION(S)

This application is based on and claims priority under 35 U.S.C. § 119 to Korean Patent Application No. 10-2020-0115302, filed on Sep. 9, 2020, in the Korean Intellectual Property Office, the disclosure of which is incorporated by reference herein its entirety.

### BACKGROUND

### 1. Field

The disclosure relates to a rotor for a centrifuge and a centrifuge including the same

### 2. Description of Related Art

A centrifuge may be used to extract peripheral blood mononuclear cells (PBMCs) or circulating tumor cell (CTCs) from blood. However, a remarkably small number of PBMCs or the CTCs are present in the blood, and, if the PBMCs or the CTCs are not separated within 24 hours after the blood of a person is collected, the cells may be destroyed. Accordingly, the PBMCs or the CTCs should be rapidly and exactly extracted.

Conventionally, after injecting a suspended density gradient material and blood into a container, such as a conical tube, and centrifuging the result, an extraction tool, such as a pipette, is inserted to a position, at which the separated PBMCs are placed, to extract the PBMCs. However, as the suspended density gradient material and the blood are mixed before the centrifugal separation, PBMCs or CTCs may be easily lost. In addition, because a person has a limitation in exactly inserting the extraction tool to the position, at which the PBMCs are placed, through a manual work, it is difficult to quantatively extract the PBMCs or the CTCs.

In addition, to extract a target cell having higher purity, secondary centrifugal separation is performed by extracting only a specific material after primary centrifugal separation. However, a worker has a limitation in exactly and rapidly transferring a material, which is primarily centrifugal-separated, to another centrifuge or another centrifugal separation chamber to perform the secondary centrifugal separation.

### SUMMARY

Aspects of the disclosure are to address at least the above-mentioned problems and/or disadvantages and to provide at least the advantages described below. Accordingly, an aspect of the disclosure is to provide a rotor for a centrifuge and a centrifuge including the same, capable of easily extracting a target material to be extracted, and exactly and rapidly transferring the extracted material for a secondary centrifuge.

In accordance with an aspect of the disclosure, a rotor for a centrifuge includes a main body to rotate about a rotating axis extending in a vertical direction and having a first space to receive a material, a first chamber coupled to the main body to be rotatable about a first axis perpendicular to a direction parallel to the vertical direction, and a second chamber coupled to the main body to be rotatable about a second axis parallel to the first axis and having a second space to receive a material. The first space and the second space have mutually different sizes, and the center of mass of the rotor is positioned on the rotating axis.

In accordance with an aspect of the disclosure, the main body may include a first opening part that is open to place the first chamber, a second opening part that is open to place the second chamber, a pair of first protrusions formed to face each other in the first axis from an inner surface of the first opening part, and a pair of second protrusions formed to face each other in a second axis from an inner surface of the second opening part. The first chamber includes a pair of first grooves to insert the pair of first protrusions, and a pair of second grooves to insert the pair of second protrusions, respectively.

In accordance with an aspect of the disclosure, the first groove and the second groove may be open in sides corresponding to the first protrusion and the second protrusion, such that the first chamber and the second chamber are coupled to the main body by putting down in the vertical direction, when the main body does not rotate about the rotating axis.

In accordance with an aspect of the disclosure, the second chamber may include an inner chamber having the second space, an outer chamber coupled to the main body to receive the inner chamber, and a balance member received in the outer chamber. The center of the mass of the rotor may be positioned on the rotating axis, as balance members having mutually different weights are selectively received in the outer chamber.

In accordance with an aspect of the disclosure, an end portion, which is at a side of the outer chamber, of the inner chamber, may be formed to be narrowed in width toward the outer chamber, and the balance member may have a hole part such that the end portion, which is at the side of the outer chamber, of the inner chamber is at least partially inserted into the hole part.

In accordance with an aspect of the disclosure, a centrifuge includes a rotor for the centrifuge and including a main body to rotate about a rotating axis extending in a vertical direction, a first chamber coupled to the main body to be rotatable about a first axis perpendicular to a direction parallel to the vertical direction and having a first space to receive a material, and a second chamber coupled to the main body to be rotatable about a second axis parallel to the first axis and having a second space to receive a material. The first space and the second space have mutually different sizes, and the center of mass of the rotor is positioned on the rotating axis.

In accordance with an aspect of the disclosure, the centrifuge may further include a tool to supply the materials to the first chamber and the second chamber, or to obtain the materials from the first chamber and the second chamber. The tool may be controlled to sequentially perform a first operation of supplying an initial material, which is to be primarily centrifugal-separated in the first chamber, to the first chamber, and a second operation of obtaining a material primarily centrifugal-separated, after the primary centrifugal separation, and supplying the material to the second chamber.

In accordance with an aspect of the disclosure, the centrifuge may further include a motor to rotate the main body about the rotating axis, and a cover to surround outer portions of the first chamber and the second chamber in a circumferential direction, when the main body rotates.

In accordance with an aspect of the disclosure, the centrifuge may further include a motor to rotate the main body about the rotating axis, a rotary union coupled to the motor and including an outer wheel coupled to the motor to rotate about the rotating axis, and an inner wheel coupled to the main body to rotate relatively to the outer wheel, a first tube connector directly or indirectly connected with the inner wheel, a second tube connector directly or indirectly connected with the outer wheel, a first tube having one end connected with a vessel having a material, and an opposite end connected with the first tube connector, and a second tube having one end connected with the second tube connector and an opposite end connected with the first chamber. When the motor operates, the outer wheel, the second tube connector, the second tube, the main body, the first chamber, and the second chamber rotate about the rotating axis, and the inner wheel, the first tube connector, and the first tube may not rotate.

In accordance with an aspect of the disclosure, the material received in the vessel may be supplied into the first chamber through the first tube, the first tube connector, the inner wheel, the outer wheel, the second tube connector, and the second tube when the main body rotates.

Other aspects, advantages, and salient features of the disclosure will become apparent to those skilled in the art from the following detailed description, which, taken in conjunction with the annexed drawings, discloses various embodiments of the disclosure.

### BRIEF DESCRIPTION OF THE DRAWINGS

The above and other aspects, features, and advantages of certain embodiments of the disclosure will be more apparent from the following description taken in conjunction with the accompanying drawings, in which:

FIG. 1 is a perspective view illustrating a centrifuge, according to an embodiment of the disclosure;

FIG. 2 is an exploded perspective view illustrating a centrifuge, according to an embodiment of the disclosure;

FIG. 3 is a perspective view illustrating a centrifuge when viewed at an angle different from that of FIG. 1, according to an embodiment of the disclosure;

FIG. 4 is a partial sectional view illustrating a centrifuge, according to an embodiment of the disclosure;

FIG. 5 is a perspective view illustrating a first chamber, according to an embodiment of the disclosure;

FIG. 6 is a perspective view illustrating a second chamber, according to an embodiment of the disclosure;

FIG. 7 is a perspective view illustrating a rotor for a centrifuge which injects a material into a first chamber through a tool, according to an embodiment of the disclosure;

FIG. 8 is a side view illustrating a centrifuge performing centrifugal separation, according to an embodiment of the disclosure;

FIG. 9 is a perspective view illustrating a rotor for a centrifuge which injects a material into a second chamber through a tool, according to an embodiment of the disclosure; and

FIG. 10 is a view illustrating a method for centrifugal-separation using a first chamber, according to an embodiment of the disclosure.

### DETAILED DESCRIPTION

Hereinafter, various embodiments of the disclosure will described with reference to accompanying drawings. However, those of ordinary skill in the art should understand that the disclosure is not limited to a specific embodiment, and modifications, equivalents, and/or alternatives on the various embodiments described herein can be variously made without departing from the scope and spirit of the disclosure. With regard to description of drawings, similar components may be assigned with similar reference numerals

In the disclosure, it will be further understood that the terms “have”, “can have,” “includes” and/or “can include”, when used herein, specify the presence of stated features (for example, components such as a numeric value, a function, an operation, or a part), but do not preclude the presence or addition of one or more other features.

In the disclosure, the expressions “A or B”, “at least one of A and/or B”, “one or more of A and/or B” may include all possible combinations of one or more of the associated listed items. For example, “A or B”, “at least one of A and B”, or “at least one of A or B” includes all (1) at least one A, (2) at least one B, or (3) at least one “A” and at least one “B”.

The wording “˜ configured to” used in the disclosure can be interchangeably used with, for example, “suitable for”, “having the capacity to”, “designed to”, “adapted to”, “made to”, or “capable of”. The wording “˜configured to” does not refer to essentially “specifically designed to”.

The terms in the disclosure are used only for specific embodiments, and the scope of another embodiment is not limited thereto. The terms of a singular form may include plural forms unless otherwise specified. In addition, unless otherwise defined, all terms used in the disclosure, including technical or scientific terms, have the same meanings as those generally understood by those skilled in the art to which the disclosure pertains. Such terms, which are used herein, as those defined in a generally used dictionary are to be interpreted as having meanings equal to the contextual meanings in the relevant field of art, and are not to be interpreted as having ideal or excessively formal meanings unless clearly defined as having such in the disclosure. Even if the terms are defined in the disclosure, the terms should not be interpreted as excluding embodiments of the disclosure if necessary.

The embodiment disclosed herein should be suggested for the convenience of explanation, and should not limit the scope of the disclosure. Accordingly, the technical scope of the disclosure should be interpreted as including all modifications or various changes based on the technical spirit of the disclosure

Hereinafter, the embodiment of the disclosure will be described in detail. Before the description of the embodiment, terms and words used in the present specification and the claims should not be interpreted as commonly-used dictionary meanings, but should be interpreted as to be relevant to the technical scope of the disclosure based on the fact that the inventor may properly define the concept of the terms to explain the disclosure in best ways

Therefore, features of the embodiment described in the disclosure are only part of the most exemplary embodiments of the disclosure, and do not represent all technical scopes of the embodiments, so it should be understood that various equivalents and modifications could exist at the time of filing this application

Throughout the whole specification, when a certain part “includes” a certain component, the certain part does not exclude other components, but may further include other components unless there is a specific opposite description

Hereinafter, the disclosure will be described in detail.

### Centrifuge

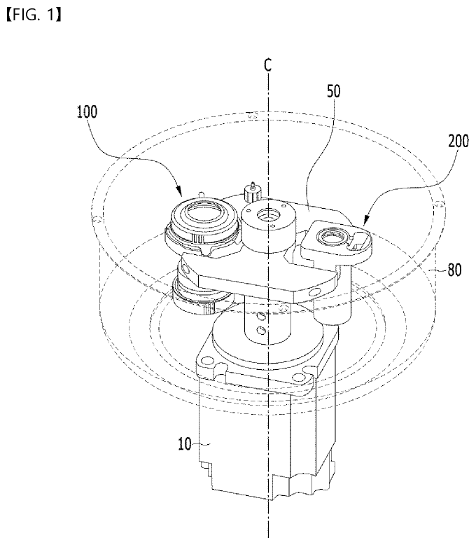

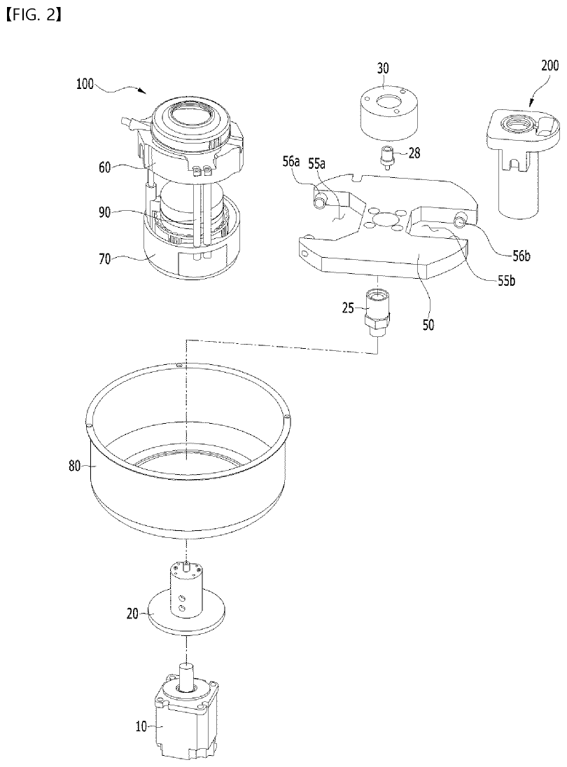

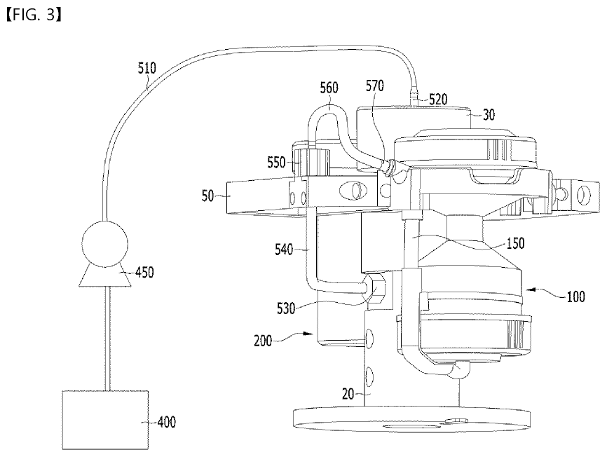

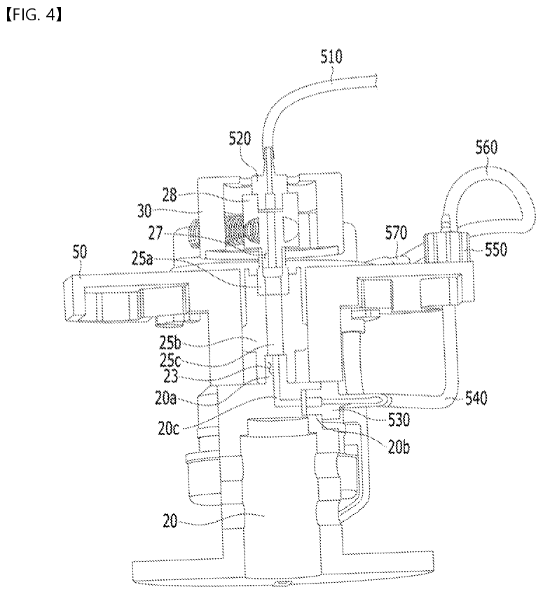

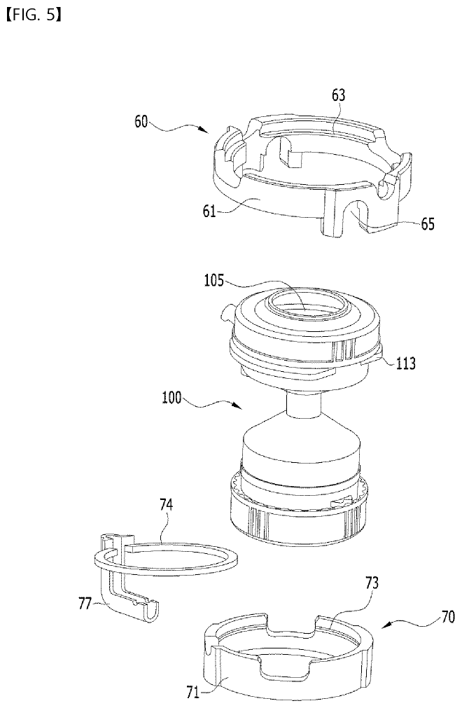

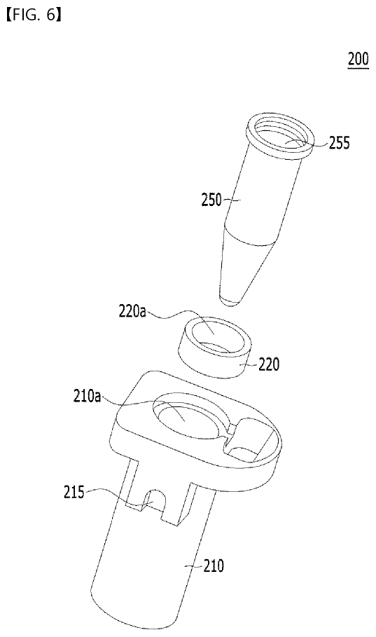

FIG. 1 is a perspective view illustrating a centrifuge, according to an embodiment of the disclosure. FIG. 2 is an exploded perspective view illustrating a centrifuge, according to an embodiment of the disclosure. FIG. 3 is a perspective view illustrating a centrifuge when viewed at an angle different from that of FIG. 1, according to an embodiment of the disclosure. FIG. 4 is a partial sectional view illustrating a centrifuge, according to an embodiment of the disclosure. FIG. 5 is a perspective view illustrating a first chamber, according to an embodiment of the disclosure. FIG. 6 is a perspective view illustrating a second chamber, according to an embodiment of the disclosure. Hereinafter, a centrifuge will be described with respect to FIG. 1 to FIG. 6.

According to an embodiment of the disclosure, a centrifuge includes a rotor for the centrifuge including a main body 50, a first chamber 100, and a second chamber 200. The main body 50 is coupled to a motor 10 to rotate about a rotating axis “C” extending in a vertical direction. In more detail, a rotary flange 20 is coupled to the motor 10, and a rotary union 25 is coupled to a distal end of the rotary flange 20 such that the rotary union 25 passes through the center of the main body 50.

Referring to FIGS. 3 and 4, the rotary union 25 typically includes an inner wheel 25a and an outer wheel 25b, and has a structure in which the inner wheel 25a and the outer wheel 25b rotate relatively to each other about the central axis “C” of the rotary union 25. According to an embodiment of the disclosure, a distal end of the inner wheel 25a of the rotary union 25 is coupled to a rotary union holder 30, and the outer wheel 25b of the rotary union 25 is fixedly coupled to the rotary flange 20 and the main body 50. Accordingly, when the motor 10 is operated, the rotary flange 20, the outer wheel 25b of the rotary union 25, the main body 50 may rotate about the central axis “C”, and the rotary union holder 30 and the inner wheel 25a of the rotary union 25 may be fixed.

An inner wheel opening part 27 is provided in one end of the inner wheel 25a of the rotary union 25, and has a structure of being connected with an outer wheel opening part 23 provided in one end of the outer wheel 25b through a duct 25c provided in the rotary union 25.

Referring to FIGS. 3 and 4, a medium connector 28 may be engaged with the inner wheel opening part 27 to connect the rotary union 25 with a first tube connector 520. In addition, one end of a tube (first tube) 510 may be coupled to the first tube connector 520, and an opposite end of the tube 510 may be coupled to various vessels 400, such as a reagent vessel, a saline vessel, and density gradient material vessel.

Meanwhile, the outer wheel opening part 23 of the rotary union 25 may be connected with a rotary flange opening part 20a provided in one end of the rotary flange 20, and the rotary flange opening part 20a may communicate with a second rotary flange opening part 20b provided in the rotary flange 20 through a duct 20c formed in the rotary flange 20. In addition, a second tube connector 530 may be coupled to the second rotary flange opening part 20b, a third tube connector 550 may be mounted on the main body 50, and a fourth tube connector 570 may be mounted on the first chamber 100. The second tube connector 530 is connected with the third tube connector 550 through a tube (a component of the second tube) 540, and the third tube connector 550 is connected with the fourth tube connector 570 through a tube (a component of the second tube) 560.

Accordingly, a fluid supplied from the vessel 400 to the tube 510 by a pump 450 may be supplied into the first chamber 100 through the tube 510, the first tube connector 520, the medium connector 28, the duct 25c, the duct 20c, the second tube connector 530, the tube 540, the third tube connector 550, the tube 560, the fourth tube connector 570, and a tube (component of the second tube) 150. In addition, the fluid may be extracted from the first chamber 100 through a reverse route. The fluid may be supplied and extracted regardless of the rotation of the rotor including the first chamber 100.

Referring to FIG. 5 and FIG. 6, the first chamber 100 and the second chamber 200 have a first space 105 and a second space 255 to receive materials, respectively. When the first chamber 100 and the second chamber 200 are coupled to the main body 50 and the main body 50 rotates about the rotating axis “C”, the materials received in the first space 105 and the second space 255 are centrifugally separated.

In more detail, the first chamber 100 and the second chamber 200 are coupled to the main body 50 to be rotatable about a first axis and a second axis, respectively. The first axis and the second axis refer to axes perpendicular to a direction parallel to a vertical direction. In other words, the second axis is substantially parallel to the first axis. Accordingly, when the main body 50 rotates about the rotating axis “C”, the first chamber 100 and the second chamber 200 receive centrifugal force to rotate about the first axis and the second axis, respectively.

The first chamber 100 and the second chamber 200 becomes in a status of being perpendicular to the rotating axis “C”, that is, in a substantially horizontal status to rotate about the rotating axis “C” while performing centrifugal separation. In addition, when the main body 50 is stopped rotating, the first chamber 100 and the second chamber 200 rotate about the first axis and the second axis again to be returned to an original status, that is, the status that the extending direction of the first chamber 100 and the second chamber 200 are substantially parallel to the rotating axis “C”.

Meanwhile, the first space 105 of the first chamber 100 and the second space 255 of the second chamber 200 have mutually different sizes. For example, the first space 105 is formed to be greater than the size of the second space 255. As the blood, or the weights of the blood and the density gradient material are previously determined and then the blood and the density gradient material are injected, to place, on the rotating axis “C”, the center of mass of a rotor including all structures coupled to the first chamber 100, the second chamber 200, and the main body 50 to rotate. Accordingly, balance may be made about the rotating axis “C” in rotating. In addition, an additional balance member 220 may be provided. Accordingly, as the main body 50 rotates, the first chamber 100 and the second chamber 200 receive centrifugal forces having substantially equal intensities. Accordingly, centrifugal-separation operations are simultaneously and smoothly performed in the first chamber 100 and the second chamber 200.

Referring to FIG. 2, the main body 50 includes a first opening part 55a and a second opening part 55b such that the first chamber 100 and the second chamber 200 are positioned in the first opening part 55a and the second opening part 55b. In addition, the main body 50 includes a pair of first protrusions 56a which protrude in the first axis from an inner surface of the first opening part 55a while facing each other, and a pair of second protrusions 56b which protrude in the second axis from an inner surface of the second opening part 55b while facing each other.

In addition, the first chamber 100 includes a pair of first grooves 65 to insert the pair of first protrusions 56a (see FIG. 5), and the second chamber 200 includes a pair of second grooves 215 to insert the pair of second protrusions 56b (see FIG. 6). In other words, as the first protrusion 56a is inserted into the first groove 65, and the second protrusion 56b is inserted into the second groove 215, the first chamber 100 and the second chamber 200 may be coupled to the main body 50 to be rotatable about the first axis and the second axis, respectively.

In addition, a cover 80 is coupled to the motor 10 or an outer portion of the rotary flange 20 to surround the first chamber 100 and the second chamber 200 during the centrifugal separation. The cover 80 has an upper opening, and has the shape of extending in the extending direction of the rotating axis “C” to surround the circumferences of the first chamber 100 and the second chamber 200. In addition, the cover 80 may be coupled to the motor 10 or the rotary flange 20 through a bearing, regardless of the rotation of the motor 10, that is, without rotating even if the motor 10 rotates.

Referring to FIG. 5, the first chamber 100 may be coupled to the main body 50 through a first holder 60 having the first groove 65. The first holder 60 includes a holder main body 61 having an annular shape, and a groove 63 formed in an inner circumferential surface of the holder main body 61. A flange 113 is formed on an upper end of the first chamber 100 while protruding in a circumferential direction. Accordingly, as the flange 113 and the groove 63 are coupled to each other, the first holder 60 may be coupled to the upper end of the first chamber 100.

In addition, the first groove 65 has the shape of opening in the side corresponding to the first protrusion 56a. Accordingly, when the main body 50 does not rotate about the rotating axis “C”, the first chamber 100 may be easily coupled to the main body 50 by putting down in the vertical direction. In other words, as a lower portion of the first groove 65 is open, the first chamber 100 and the main body 50 may be easily coupled to each other and the first chamber 100 may rotate in the direction perpendicular to the rotating axis “C” by receiving the centrifugal force during the centrifugal separation.

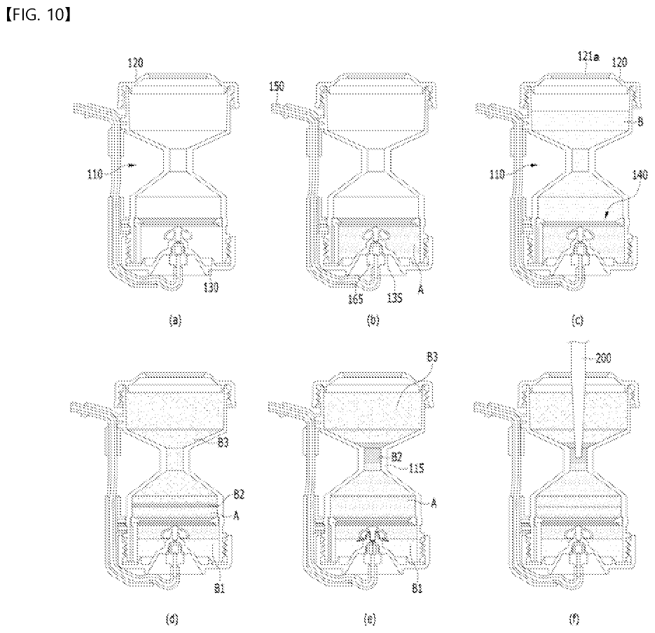

Meanwhile, a second holder 70 is coupled to the lower portion of the first chamber 100. The second holder 70 may support a tube holder 77. The tube holder 77 is a component to fix a tube 150 (see FIG. 10) which supplies a material into the first chamber 100. To this end, the second holder 70 includes a holder main body 71 having an annular shape, and a groove 73 formed in an inner circumferential surface of the holder main body 71. A ring 74 having an annular shape and formed on the tube holder 77 is fitted into the groove 73. In the status that the tube holder 77 is coupled to the holder main body 71, the second holder 70 may be coupled to the lower portion of the first chamber 100.

In addition, referring to FIG. 2, according to an embodiment of the disclosure, the centrifuge may include a plurality of rods 90 to couple the first holder 60 with the second holder 70. During centrifugal separation, the cover 80 coupled to the lower portion of the first chamber 100 may be separated by the centrifugal force. However, the above-described second holder 70 may prevent the cover 80 from being separated, and the rod 90 couples the second holder 70 to the first holder 60, thereby more preventing the cover 80 from being separated.

Referring to FIG. 6, the second chamber 200 includes an inner chamber 250, an outer chamber 210, and the balance member 220. The inner chamber 250 has the second space 255 and is received in an inner space 210a of the outer chamber 210. The outer chamber 210 is coupled to the main body 50, and has the second groove 215. The second groove 215 is formed to be open in the side corresponding to the second protrusion 56b, similar to the first groove 65, such that the outer chamber 210 is easily coupled to the main body 50.

The balance member 220 is received in the outer chamber 210, and interposed between the outer chamber 210 and the inner chamber 250. Balance members 220 having mutually different weights are selectively received in the outer chamber 210 such that the center of mass of a rotor including all structures coupled to the first chamber 100, the second chamber 200, and the main body 50 is positioned on the rotating axis “C”.

An end portion, which is at the side of the outer chamber 210, of the inner chamber 250, is formed to be narrowed in width toward the outer chamber 210. In addition, the balance member 220 has a hole part 220a such that the end portion, which is at the side of the outer chamber 210, of the inner chamber 250 is at least partially inserted into the hole part 220a. In the status that the inner chamber 250 is inserted into the hole part 220a, the inner chamber 250 is received in the outer chamber 210, such that the inner chamber 250 and the balance member 220 are stably received in the outer chamber 210. The hole part 220a may be formed through the balance member 220.

### Method for Centrifugal Separation

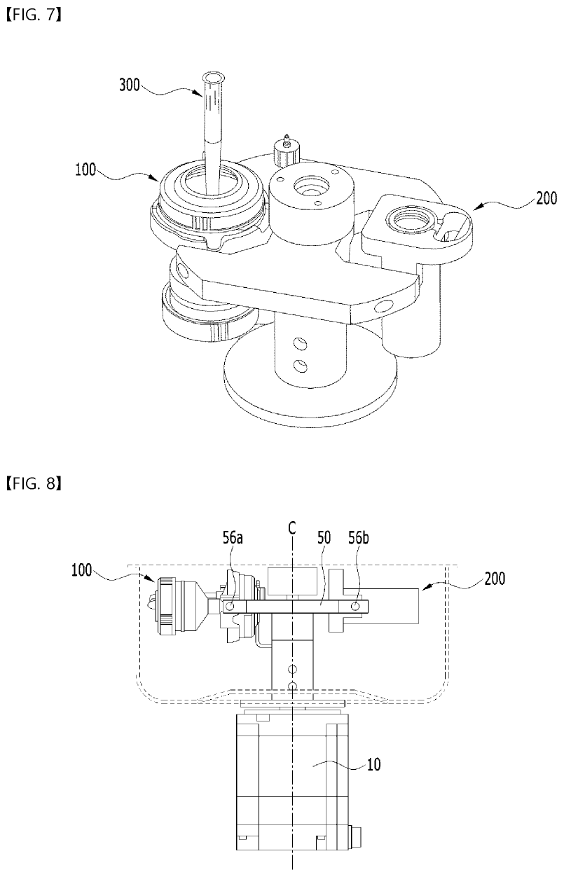

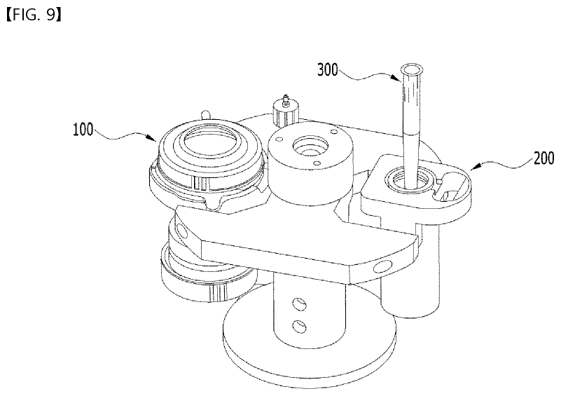

FIG. 7 is a perspective view illustrating a rotor for a centrifuge which injects a material into a first chamber through a tool, according to an embodiment of the disclosure. FIG. 8 is a side view illustrating a centrifuge performing centrifugal separation, according to an embodiment of the disclosure. FIG. 9 is a perspective view illustrating a rotor for a centrifuge which injects a material into a second chamber through a tool, according to an embodiment of the disclosure. FIG. 10 is a view illustrating a method for centrifugal-separation using a first chamber, according to an embodiment of the disclosure.

Hereinafter, a method for centrifugal separation will be described with respect to FIG. 7 to FIG. 10, according to an embodiment of the disclosure. The following description will be made regarding a manner of extracting a PBMC from blood by performing the centrifugal separation with respect to the blood. However, a target material for centrifugal separation and a target material to be extracted are not specifically limited.

First, referring to FIG. 7, according to an embodiment of the disclosure, a centrifuge is prepared. The centrifuge includes the first chamber 100 and the second chamber 200. According to an embodiment of the disclosure, the centrifuge may further include the tool 300 to supply a material to the first chamber 100 and the second chamber 200 or to extract and obtain a material from the first chamber 100 and the second chamber 200.

When the centrifuge is prepared, a density gradient material “A” is injected into the first chamber 100 (see reference signs (a) and (b) of FIG. 10). The first chamber 100 includes a first chamber main body 110, and a first cover 120 and a second cover 130 coupled to the first chamber main body 110. The tube 150 is coupled to the first chamber main body 110. The density gradient material “A” may be supplied to the first chamber 100 along a route formed through tubes 510, 540, 560, and 150 (see FIGS. 3 and 4). In addition, the second cover 130 may protrude inward of the first chamber main body 110, and a protruding part 135 having a second injection port may be formed. The density gradient material “A” supplied to the tube 150 is injected into the first chamber main body 110 through the second injection port after passing through a tube connector 165 mounted on the protruding part 135 (see reference sign (b) of FIG. 10).

Next, blood “B” is injected through a first injection port 121a provided in the first cover 120 (see reference sign (c) of FIG. 10). As illustrated in FIG. 7, the blood “B” may be injected by inserting the tool 300, such as a syringe or a pipette, into the first injection port 121a. In this case, a filter device 140 is provided in the first chamber main body 110, thereby preventing the blood “B” and the density gradient material “A” from being mixed with each other.

The centrifugal separation is performed in the above status (see FIG. 7). When the motor 10 is operated, the first chamber 100 and the second chamber 200 receive the centrifugal force to rotate about axes (the first axis and the second axis) passing through the first protrusion 56a and the second protrusion 56b, and to perform the centrifugal separation while being perpendicular to the rotating axis “C”. Accordingly, plasma “B3”, PBMC “B2”, the density gradient material “A”, and red blood cell “B1” are separated and sequentially positioned (see reference sign (d) of FIG. 10).

In this status, the density gradient material “A” is injected again into the first chamber main body 110 (see reference sign (e) of FIG. 10). The first chamber main body 110, which includes a connecting unit 115 having a smaller sectional area in a horizontal direction, supplies the density gradient material “A” to lift the PBMC “B2” to the position of the connecting unit 115 such that the PBMC “B2”, which serves as the target material to be extracted, is easily extracted. In this case, because a cap 170 is coupled to the second injection port, the density gradient material “A”, which is supplied again, is prevented from moving toward the first injection port 121a. Accordingly, even if the density gradient material “A” is injected again, the plasma “B3”, the PBMC “B2”, the density gradient material “A”, and the red blood cell “B1” are prevented from being mixed with each other. In addition, the filter device 140 may prevent the separated ingredients from being mixed with each other again.

When the PBMC “B2” is placed at the position of the connecting unit 115, the tool 300 is controlled to be inserted into the position of the connecting unit 115 through the first injection port 121a to extract the PBMC “B2” (see reference sign (f) of FIG. 10). The procedure of lifting the PBMC “B2” till the position of the connecting unit 115 by injecting the density gradient material “A” again may make the target material conveniently and exactly extracted merely through a control work of inserting the tool 300 till the position of the connecting unit 115.

Referring to FIG. 9, the tool 300 is controlled such that the extracted PBMC “B2” is supplied to the second chamber 200. The material extracted after the primary centrifugal separation may contain other materials such as a buffy coat, as well as PBMC “B2”. In other words, because the extracted PBMC “B2” has no purity in a desired extent, the secondary centrifugal separation may be required. Accordingly, the material (intermediate material) extracted from the first chamber 100 is controlled to be automatically supplied to the second chamber 200. When the intermediate material is supplied to the second chamber 200, the secondary centrifugal separation is performed as illustrated in FIG. 8, such that a target cell having higher purity is separated.

As described above, because the center of the mass of the rotor is positioned on the rotating axis “C”, the centrifugal separation is simultaneously and smoothly performed in the first chamber 100 and the second chamber 200. Accordingly, while the secondary centrifugal separation is performed in the second chamber 200, primary centrifugal separation for a new material may be performed in the first chamber 100.

In addition, because the second space 255 of the second chamber 200 is larger than the first space 105 of the first chamber 100, the target cell having higher purity may be separated from a smaller amount of intermediate materials, concentrated, and extracted. Accordingly, the purity of the target cell may be enhanced.

In addition, the tool 300 is controlled to sequentially or automatically perform a first operation of supplying an initial material, which is to be primarily centrifugal-separated in the first chamber 100, to the first chamber 100, and a second operation of obtaining a material primarily centrifugal-separated, after the primary centrifugal separation, and supplying the material to the second chamber 200. Accordingly, the intermediate material, which is centrifugal-separated in the first chamber 100, is automatically extracted by the tool 300 and supplied to the second chamber 200. Accordingly, the target cell may be more exactly and rapidly extracted, as compared to when the target cell is extracted by a person.

According to the rotor for the centrifuge of the disclosure, the first space and the second space have mutually different sizes, and the center of the mass is positioned on the rotating axis, thereby performing secondary centrifugal separation for a material, which is extracted through the primary centrifugal separation in the first chamber, in the second chamber, such that the target cell is separated, concentrated, and extracted.

In addition, according to the centrifuge of the disclosure, the primary centrifugal separation may be performed in the first chamber, and the intermediate material, which is primarily centrifugal-separated, may be automatically performed in the second chamber, thereby exactly and rapidly improving the target cell.

Hereinabove, although the disclosure has been described with reference to exemplary embodiments and the accompanying drawings, the disclosure is not limited thereto, but may be variously modified and altered by those skilled in the art to which the disclosure pertains without departing from the spirit and scope of the disclosure claimed in the following claims. Therefore, the embodiments of the disclosure are provided to explain the spirit and scope of the disclosure, but not to limit them, so that the spirit and scope of the disclosure is not limited by the embodiments. The scope of the disclosure should be construed on the basis of the accompanying claims, and all the technical ideas within the scope equivalent to the claims should be included in the scope of the disclosure.

What is claimed is:

1. A rotor for a centrifuge, the rotor comprising:
a main body configured to rotate about a rotating axis extending in a vertical direction;
a first chamber coupled to the main body to be rotatable about a first axis perpendicular to a direction parallel to the vertical direction, and having a first space to receive a material; and
a second chamber coupled to the main body to be rotatable about a second axis parallel to the first axis, and having a second space to receive a material,
wherein the first space and the second space have mutually different sizes, and the center of mass of the rotor is positioned on the rotating axis.

2. The rotor of claim 1, wherein the main body includes:
a first opening part that is open to place the first chamber;
a second opening part that is open to place the second chamber;
a pair of first protrusions formed to face each other while extending in the first axis from an inner surface of the first opening part; and
a pair of second protrusions formed to face each other while extending in the second axis from an inner surface of the second opening part, and
wherein the first chamber includes:
a pair of first grooves to insert the pair of first protrusions, respectively; and
a pair of second grooves to insert the pair of second protrusions, respectively.

3. The rotor of claim 2, wherein the first groove and the second groove are open in sides corresponding to the first protrusion and the second protrusion, such that the first chamber and the second chamber are coupled to the main body by putting down in the vertical direction, when the main body does not rotate about the rotating axis.

4. The rotor of claim 1, wherein the second chamber includes:
an inner chamber having the second space;
an outer chamber coupled to the main body to receive the inner chamber; and
a balance member received in the outer chamber, and
wherein the center of the mass of the rotor is positioned on the rotating axis, as one of balance members having mutually different weights is selectively received in the outer chamber.

5. The rotor of claim 4, wherein an end portion, which is at a side of the outer chamber, of the inner chamber is formed to be narrowed in width toward the outer chamber, and
wherein the balance member has a hole part such that the end portion, which is at the side of the outer chamber, of the inner chamber is at least partially inserted into the hole part.

6. A centrifuge comprising:
a rotor for the centrifuge,
wherein the rotor includes:
a main body to rotate about a rotating axis extending in a vertical direction;
a first chamber coupled to the main body to be rotatable about a first axis perpendicular to a direction parallel to the vertical direction, and having a first space to receive a material; and
a second chamber coupled to the main body to be rotatable about a second axis parallel to the first axis, and having a second space to receive a material, and
wherein the first space and the second space have mutually different sizes, and the center of mass of the rotor is positioned on the rotating axis.

7. The centrifuge of claim 6, further comprising:
a tool to supply the materials to the first chamber and the second chamber, or to obtain the materials from the first chamber and the second chamber,
wherein the tool is controlled to sequentially perform a first operation of supplying an initial material, which is to be primarily centrifugal-separated in the first chamber, to the first chamber, and a second operation of obtaining a material primarily centrifugal-separated, after the primary centrifugal separation is performed, and supplying the material to the second chamber.

8. The centrifuge of claim 6, further comprising:
a motor to rotate the main body about the rotating axis; and
a cover to surround outer portions of the first chamber and the second chamber in a circumferential direction, when the main body rotates.

9. The centrifuge of claim 6, further comprising:
a motor to rotate the main body about the rotating axis;
a rotary union coupled to the motor and including an outer wheel coupled to the motor to rotate about the rotating axis, and an inner wheel coupled to the main body to rotate relatively to the outer wheel;
a first tube connector directly or indirectly connected with the inner wheel;
a second tube connector directly or indirectly connected with the outer wheel;
a first tube having one end connected with a vessel to receive the material, and an opposite end connected with the first tube connector; and
a second tube having one end connected with the second tube connector and an opposite end connected with the first chamber, and
wherein the outer wheel, the second tube connector, the second tube, the main body, the first chamber, and the second chamber rotate about the rotating axis, and the inner wheel, the first tube connector, and the first tube do not rotate, when the motor operates, when the motor operates.

10. The centrifuge of claim 9, wherein the material received in the vessel is supplied into the first chamber through the first tube, the first tube connector, the inner wheel, the outer wheel, the second tube connector, and the second tube, when the main body rotates.
